(19) United States
(12) Patent Application Publication
(10) Pub. No.: US 2022/0175868 A1
(43) Pub. Date: Jun. 9, 2022

(54) INHIBITION OF SARS-CoV-2 VIRUS BY ARUM PALAESTINUM

(71) Applicant: Ankh Life Sciences Limited, Dublin 2 (IE)

(72) Inventors: Gene H. Zaid, Hutchinson, KS (US); Beth Ann Wolf, Hutchinson, KS (US); Robert Preston Moore, Great Bend, KS (US); Larry D. Crow, Hutchinson, KS (US); Jason H. West, Hutchinson, KS (US)

(73) Assignee: Ankh Life Sciences Limited, Dublin 2 (IE)

(21) Appl. No.: 17/540,766

(22) Filed: Dec. 2, 2021

Related U.S. Application Data
(60) Provisional application No. 63/121,758, filed on Dec. 4, 2020.

(51) Int. Cl. A61K 36/888; A61P 31/14

(52) U.S. Cl. CPC A61K 36/888; A61K 2236/333; A61P 31/14

(57) ABSTRACT

The treatment of human subjects exposed to, or potentially exposed to SARS-CoV-2 virus, or suffering from COVID-19, comprises the step of administering to the human subjects a formulation comprising Arum spp., particularly Arum palaestinum. Methods of inhibiting SARS-CoV-2 viral replication are also disclosed comprising contacting the virus with an Arum formulation.

### CROSS-REFERENCE TO RELATED APPLICATIONS

The present application claims the priority benefit of U.S. Provisional Patent Application Ser. No. 63/121,758, filed Dec. 4, 2020, entitled INHIBITION OF SARS-CoV-2 VIRUS BY ARUM PALAESTINUM, incorporated by reference in its entirety herein.

### BACKGROUND OF THE INVENTION

### Field of the Invention

The present invention is broadly concerned with a new medicament for inhibition of SARS-CoV-2 virus, the cause of the ongoing COVID-19 pandemic. More particularly, the invention is concerned with the use of formulations comprising Arum spp., preferably Arum palaestinum, as effective agents inhibiting replication of the virus and the resulting COVID-19 disease.

### Description of the Prior Art

The COVID-19 pandemic, also known as the coronavirus pandemic, is an ongoing pandemic of coronavirus disease 2019 (COVID-19) caused by severe acute respiratory syndrome coronavirus 2 (SARS-CoV-2), first identified in December 2019 in Wuhan, China. COVID-19 mainly spreads through the air when people are near each other long enough, primarily via small droplets or aerosols, as an infected person breathes, coughs, sneezes, sings, or speaks.

Transmission via fomites (contaminated surfaces) has not been conclusively demonstrated. It can spread as early as two days before infected persons show symptoms (presymptomatic), and from asymptomatic (no symptoms) individuals. People remain infectious for up to ten days in moderate cases, and two weeks or longer in severe cases.

Common symptoms include fever, cough, fatigue, breathing difficulties, and loss of smell and taste. Complications may include pneumonia and acute respiratory distress syndrome. The incubation period is typically around five days but may range from one to 14 days.

The responses have caused global social and economic disruption, including the largest global recession since the Great Depression. It has led to the postponement or cancellation of events, widespread supply shortages exacerbated by panic buying, famines affecting hundreds of millions of people, and decreased emissions of pollutants and greenhouse gases. Educational institutions have been partially or fully closed. Misinformation has circulated through social media and mass media.

In response to these conditions, there has been a tremendous scientific effort to develop both prophylactic vaccines and medication treatments. Despite the development of several vaccines, and emerging treatment protocols, transmission and infection remains high, as the virus mutates into different forms (e.g., so-called Delta variant) evading current protocols. Thus, there remains a need in the art for new and effective treatments which inhibit the replication of SARS-CoV-2 virus, reduce viral transmission, and prevent or ameliorate symptoms of COVID-19.

Arum species, particularly Arum palaestinum has a long history of diverse clinical applications, including the treatment of infections, bone fractures, and cancer. See, for example, Said et al. Ethnopharmacological Survey of Medicinal Herbs in Israel, the Golan Heights and the West Bank Region. J. Ethnopharmacology. 83 (2002): 251-265. In use, Arum plant parts, including roots, stems, and leaves, are often boiled in water to remove toxicity that may be present in the raw form of the plant, whereupon the plant parts are dried and ground, or formulated as liquids. The portfolio of chemical constituents in Arum palaestinum includes phytochemicals, such as flavonoids, phenolic acids and their derivatives, and turpenoid derivatives. See, U.S. Pat. No. 8,039,025, incorporated by reference herein.

### SUMMARY OF THE INVENTION

The present invention provides important formulations useful for inhibiting SARS-CoV-2 virus and in the treatment of COVID-19 disease. Generally, the formulations comprise Arum palaestinum, and may also include additional ingredients. The methods involve the administration of the formulations to a subject in prophylactically or therapeutically effective amounts which inhibit replication of the SARS-CoV-2 virus in the subject.

The use of the formulations in the manufacture of a medicament for such treatment is also within the ambit of the invention.

The present disclosure also includes methods of inhibiting viral replication by contacting SARS-CoV-2 virus with Arum palaestinum in an effective amount and for an effective period of time in order to inhibit the growth, replication, and/or viability of the virus. Such usages would typically be in vitro.

### DETAILED DESCRIPTION OF THE PREFERRED EMBODIMENTS

Methods described herein include those for inhibiting replication of coronavirus, and prevention or amelioration of COVID-19. In one aspect, the methods comprise (consist essentially or even consist of) contacting coronavirus with amounts of Arum palaestinum, effective to inhibit viral replication for an effective period of time. In another aspect, the methods comprise (consist essentially or even consist of) administering therapeutically or prophylactically effective amounts of Arum palaestinum to a human subject in need thereof. The subject may be at risk of viral infection or exposure to coronavirus and/or may be an individual identified as having been infected with coronavirus. Such individual may be symptomatic or asymptomatic. Thus, “therapeutic” use of the formulation refers to processes that are intended to produce a beneficial change in an existing condition (e.g., viral infection) of a subject, such as by reducing the severity of the clinical symptoms and/or effects of the infection, and/or reducing the duration of the infection/symptoms/effects. Likewise, “prophylactic” use of the formulation refers to processes that are intended to inhibit or ameliorate the effects of a future viral infection to which a subject may be exposed (but is not currently infected with). In some cases, the formulations may prevent the development of observable morbidity from viral infection (i.e., near 100% prevention). In other cases, the formulations may only partially prevent and/or lessen the extent of morbidity due to the viral infection (i.e., reduce the severity of the symptoms and/or effects of the infection, and/or reduce the duration of the infection/symptoms/effects). In either case, the formulations are still considered to “prevent” the target infection.

### The Formulations of the Invention

In their broadest aspects, the formulations used in the context of the invention comprise (or consist essentially of, or consist of) Arum spp., preferably Arum palaestinum, as the principal ingredient serving to provide health benefits. Other ingredients, such as adjuvants, carriers, vehicles, or dispersants may also be used. Among the active agents, the Arum spp., preferably Arum palaestinum, comprises respective quantities of β-sitosterol, isovanillin, and linolenic acid. In preferred forms, the invention involves the administration of a dosage form prepared using plant parts of Arum spp., preferably Arum palaestinum Boiss, which is native to the Middle East, and particularly Palestine and adjoining regions. This plant is of the genus Arum, family Araceae, subfamily Aroideae, and tribe Areae, and has a Nomen No. of 4357. The name was verified on Nov. 5, 1985 by ARS Systematic Botanists. The species priority site is the Ornamental Plant Germplasm Center. The plant is also known by common names, including Black Calla and Solomon's-Lily.

Dosage forms can be in liquid decoction forms or solid forms. Dosage formulations can be prepared using either fresh or dried Arum plant parts. In one or more embodiments, fresh Arum bulb or other plant parts (stems, leaves, etc.) are macerated into a pulp and extracted with aqueous ethanol and/or water. In one or more embodiments, the macerated plant parts are first subjected to air drying before extraction. In one or more embodiments, the macerated plant parts are first baked, followed by grinding into a powder before extraction. In one or more embodiments, the complete plant parts are first dried and then pulverized into a powder, which can be used directly or first extracted. It will be appreciated that it is not essential that all of the plant parts be used, i.e., use may be made of the leaves, and/or stems, and/or bulbs. Regardless, the resulting extracts can then be suspended in aqueous ethanol, centrifuged, and filter sterilized to yield the resulting dosage forms. If desired, the cooled mixture can be lyophilized (freeze-dried) to obtain a dried extract. This extract may be then put in capsule form or may be tableted to provide solid dosage forms.

In one or more embodiments, solid dosage form powders should be of a size to pass through a 50-mesh screen while being retained by a 100-mesh screen. The capsules or tablets should each contain from about 20 mg to about 75 mg of the Arum, preferably from about 30 mg to about 60 mg, even more preferably from about 40 mg to about 60 mg. In one or more embodiments, daily dosages could range from about 20 mg to about 200 mg per day, preferably from about 40 mg to about 160 mg per day. In one or more embodiments, a unit dosage firm can comprise about 50 mg of Arum per capsule, with a recommended daily dosage of 1 to 4 capsules per day (50 mg to about 200 mg).

Advantageously, all of the ingredients of the formulations are at least food grade in purity. While such powdered formulations are preferred for ease of manufacture and administration, it should be understood that the invention is not so limited. For example, the Arum spp. may be prepared as liquid dispersions or solutions using appropriate, non-interfering dispersants or solvents; other possible dosage forms include gels, suspensions, or solids such as tablets or pills. Dosage forms of the invention may likewise employ a decoction or tea using plant parts (preferably leaves and/or roots) of Arum palaestinum Boiss, or any other suitable plant parts of the genus Arum steeped in boiling water.

Without wishing to be bound by theory, it is believed that the individual chemical compounds present in the Arum, including β-sitosterol, isovanillin, and linolenic acid, act in synergy to contribute to the therapeutic efficacy of the Arum dosage forms. Examples of compounds provided via the Arum dosage form includes any combinations of the following components identified via Gas Chromatography-Mass Spectroscopy (GC-MS) analysis of Arum palaestinum Boiss extract.

GCMS RESULTS

| | | Relative |
|---|---|---|
| Molecule | Formula | Amount |
| hexadecanoic acid | C16H32O2 | 1 |
| linolenic acid | C18H30O2 | 0.48 |
| linoleic acid | C18H32O2 | 0.44 |
| oleamide | C18H35NO | 0.12 |
| 2-monopalmitin | C19H38O4 | 0.17 |
| phytol | C20H40O | 0.49 |
| campesterol | C28H48O | 0.79 |
| sitostenone | C29H48O | 0.085 |
| stigmasterol | C29H48O | 0.29 |
| isofucosterol | C29H48O | 0.13 |
| 5a-stigmastane-3,6-dione | C29H48O2 | 0.038 |
| beta-sitosterol | C29H50O | 1.9 |
| cycloartenol | C30H50O | 0.19 |
| dl-a-tocopherol | C29H50O2 | 0.050 |
| heneicosane | C21H44 | 0.028 |
| tricosane | C23H48 | 0.093 |
| pentacosane | C25H52 | 0.29 |
| heptacosane | C27H56 | 0.88 |
| nonacosane | C29H60 | 2.1 |
| hentriacontane | C31H64 | 0.39 |
| 5,5,8a-Trimethyl-3,5,6,7,8,8a- | C12H20O | 0.15 |
| hexahydro-2H-chromene | | |
| 6-(3,3-Dimethyl-oxiran-2-ylidene)- | C12H18O2 | 0.029 |
| 5,5-dimethyl-hex-3-en-2-one | | |
| 2-butanone, 4(2,6,6-trimethyl-1,3 | C13H20O | 0.070 |
| cyclohexadien-1-yl) | | |
| 2-cyclohexen-1-one, 4-(3-hydroxy- | C13H20O2 | 0.042 |
| 1-butenyl)-3,5,5-trimethyl | | |
| 2-cyclohexen-1-one, 4-(3- | C13H22O2 | 0.12 |
| hydroxybutyl)-3,5,5-trimethyl- | | |
| 6-(3-Hydroxy-but-1-enyl)- | C13H22O3 | 0.17 |
| 1,5,5-trimethyl-7- | | |
| oxabicyclo[4.1.0]heptan-2-ol | | |
| 3-Buten-2-one, 4-(4-hydroxy- | C13H20O3 | 0.05 |
| 2,2,6-trimethyl-7- | | |
| oxabicyclo[4.1.0]hept-1-yl)- | | |
| isovanillin | C8H8O3 | 0.012 |
| cinnamic acid | C9H8O2 | 0.018 |
| 2 methoxy 4 vinylphenol | C9H10O2 | 0.035 |
| 2-propenal, 3-(4-hydroxy- | C10H10O3 | 0.063 |
| 3-methoxyphenyl) | | |
| docosyl hexadecanate | C16H33O2—C22H44 | 0.081 |

Fortified dosage forms are also contemplated herein, wherein one or more of the foregoing components is artificially increased in quantity by supplementing the dosage forms with additional purified amounts of such component. In preferred practice, the individual components are naturally or synthetically derived, and should have purities of at least about 90% by weight, and most preferably at least about 98% by weight.

### Use of the Formulations

In use, therapeutically effective amounts of the Arum formulation are administered to a mammalian subject in need thereof for a therapeutically effective amount of time. As used herein, a “therapeutically effective” amount refers to the dosage amount and/or duration that will elicit the biological or medical response of a tissue, system, or subject that is being sought by a researcher or clinician, and in particular elicit some desired therapeutic effect as against the coronavirus cells by slowing and/or inhibiting activity, growth, or replication of the cells. One of skill in the art recognizes that an amount or duration may be considered therapeutically “effective” even if the condition is not totally eradicated or prevented, but it or its symptoms and/or effects are improved or alleviated partially or inhibited from worsening in the subject. Such therapeutically effective dosages and durations may comprise a single unit dosage or, more usually, periodic (e.g., daily or weekly) administration of lower dosages over time.

The formulations of the invention, in whatever physical form, are designed for administration to humans, in particular those exposed, or potentially exposed, to the SARS-CoV-2 virus, or those suffering from the COVID-19 disease. The formulations may be administered in any convenient manner, such as by oral, rectal, nasal, ophthalmic, parenteral (including intraperitoneal, gastrointestinal, intrathecal, intravenous, cutaneous (e.g., dermal patch), subcutaneous (e.g., injection or implant), or intramuscular) routes. In one or more embodiments, the dosage formulations are administered in repeated dosages, e.g., daily, multiple time daily, and the like. Where the aforementioned capsules are used, the administration would be oral, and the recommended dosage level would be four such capsules per day, taken twice daily, two capsules per serving. Dosage forms may be administered with a meal, or suspended or dissolved in PEPTAMEN® or other fat-containing liquid before administration, or co-administered with such fatty substances. A clinician or researcher may determine the appropriate administration protocol depending upon the dosage form and route of administration used.

The Arum formulation may be administered in conjunction with other recommended therapies for treating COVID-19 or its symptoms. The Arum formulation may also be taken prophylactically, such as during period of time where there is high community transmission of SARS-CoV-2. The methods can be also applied for clinical research and/or study.

Additional advantages of the various embodiments of the invention will be apparent to those skilled in the art upon review of the disclosure herein. It will be appreciated that the various embodiments described herein are not necessarily mutually exclusive unless otherwise indicated herein. For example, a feature described or depicted in one embodiment may also be included in other embodiments, but is not necessarily included. Thus, the present invention encompasses a variety of combinations and/or integrations of the specific embodiments described herein.

The present description also uses numerical ranges to quantify certain parameters relating to various embodiments of the invention. It should be understood that when numerical ranges are provided, such ranges are to be construed as providing literal support for claim limitations that only recite the lower value of the range as well as claim limitations that only recite the upper value of the range. For example, a disclosed numerical range of about 10 to about 100 provides literal support for a claim reciting “greater than about 10” (with no upper bounds) and a claim reciting “less than about 100” (with no lower bounds).

Levels of dosing to human subjects of the formulations hereof are quite variable owing to factors such as the patient's age, patient's physical condition, and the severity of the disease. In general, however, regardless of the dosage form or route of administration employed, the formulations should be dosed of from about 5 to 2000 mg per day, and more usually from about 20 mg to about 200 mg per day. Such dosages may be based on a single administration per day, but more usually multiple administrations per day.

As used herein, the term “inhibit” refers to a reduction or decrease viral titer or quantity, compared to a baseline. For example, in the context of the present invention, inhibition of viral replication refers to a decrease in amount or speed of viral replication as compared to baseline (e.g., as detected by a rapid antigen test, or molecular/PCR test, or other suitable testing methodology). By comparing a baseline obtained before administration of the Arum formulation to the values obtained from the individual after administration of the Arum formulation, those of ordinary skill in the art can readily determine whether or not viral replication has been inhibited and to what extent. Thus, an “effective amount” to inhibit viral replication refers to the amount of a given formulation that results in a reduced level of viral replication and thus a reduced amount of detectable virus in the individual (e.g., reduced viral titer or viral load) when comparing the baseline detected amount to the reduced amount using the same testing methodology. Preferably, prophylactic and/or therapeutic methods of the invention will lead to a decrease of at least 20%, at least 30%, at least 40%, at least 50%, at least 60%, at least 70%, at least 80%, or even at least 90% as compared to the baseline. Preferably, such reductions are seen within 72 hours after administration, preferably within 48 hours, preferably within 36 hours, more preferably within 24 hours. Correspondingly, such reductions in viral load will advantageously lead to an amelioration, improvement, or decrease in one or more symptoms associated with coronavirus infection and/or reduced transmission of the virus from the infected individual. Alternatively, inhibition or antiviral efficacy can be assessed in vitro. In one or more embodiments, the Arum formulation will inhibit viral replication by at least 50% in a cell-based assay, preferably by at least 60%, 70%, 80%, or 90% in a cell-based assay. Preferably, the Arum formulation will have a Selectivity Index of greater than 1, preferable of at least 3, more preferably at least 10, even more preferably at least 30. The Selectivity Index is the ratio of the toxic concentration of a formulation against its effective bioactive amount. In one or more embodiments, the toxic concentration is the dose that leads to 50% cell cytotoxicity in an in vitro cell assay. In one or more embodiments, the bioactive amount is the dose that inhibits viral replication by 90% in a cell assay.

Furthermore, the phrase “and/or,” when used in a list of two or more items, means that any one of the listed items can be employed by itself or any combination of two or more of the listed items can be employed. For example, if a formulation is described as containing or excluding components A, B, and/or C, the formulation can contain or exclude A alone; B alone; C alone; A and B in combination; A and C in combination; B and C in combination; or A, B, and C in combination.

### EXAMPLES

The following examples set forth methods in accordance with the invention. It is to be understood, however, that these examples are provided by way of illustration and nothing therein should be taken as a limitation upon the overall scope of the invention.

In Vitro Assay of COVID 19 Using Arum palaestinum

The antiviral activity of Arum palaestinum on coronavirus was assessed in vitro using novel coronavirus SARS-CoV-2 isolate USA-WA1/2020 (deposited by the Centers for Disease Control and Prevention). For propagation and experimentation with SARS-CoV-2, we used Vero E6 (ATCC® CRL-1586™) cells maintained in EMEM (Eagle's Minimum Essential Medium) cell culture media (cat #30-2003, ATCC, Manassas, Va., USA) supplemented with 2% or 10% fetal bovine serum (FBS), 100 U/mL penicillin, 100 μg/mL streptomycin, 0.01M HEPES buffer solution, 1 mM sodium pyruvate, lx non-essential amino acids solution (cat #SH3023801, Thermo Fisher Scientific, Waltham, Mass., USA), and 2 mM L-glutamine.

Several different test formulations were prepared. Two different formulations were prepared from “fresh” Arum palaestinum bulb, which was macerated into a pulp and extracted with either 70% or 20% EtOH. Two additional formulations were prepared from “dried” Arum palaestinum bulb by first macerating the bulb and then subjecting the pulp to air drying before extracting with either 20% EtOH or water (0% EtOH). Finally, three formulations were prepared by extracting “baked” Arum palaestinum powder with either 70% EtOH, 20% EtOH, or water (0% EtOH). The resulting extracts were then suspended in aqueous ethanol in a 1:10 wt:volume ratio, centrifuged and filter sterilized.

Plated cells were first infected with SARS-CoV2 at a multiplicity of infection (MOI) of 0.001. A range of doses of the Arum palaestinum formulations were added during and post-infection. At 72 hours post-infection, the cell monolayers were stained with crystal violet to determine the level of cytopathic effect (CPE).

The CPE level was quantified to determine the IC₉₀ (dose that inhibited viral replication by 90%). For the antiviral assay, we went as low as 0.1 μl/ml but found the IC₉₀ at 0.5 μl/ml (7 μg/ml). Vero cells were also treated with varying doses of the test formulation alone for 24 hours to determine the CC₅₀ (dose that leads to 50% cell cytotoxicity). For the CC₅₀ tests, dosage went up to 200 μl/ml (2800 μg/ml) and still did not achieve cell death; this was the upper limit of what could practically be added to the media. After 24 hours of treatment, cell viability was determined by a standard MTS colorimetric assay.

Based on the IC₉₀ and CC₅₀, the Selectivity Index (CC₅₀/IC₉₀) of each of the treatments could be determined. A higher Selectivity Index indicates that the treatment will, theoretically, be both safter and more effective during in vivo treatment for a given viral infection. The results are in the table below.

TABLE

| | CC₅₀ | IC₉₀ | Selectivity |
|---|---|---|---|
| Test Formulation | (μl/ml) | (μl/ml) | Index |
| A. palaestinum, 70% EtOH, | 55 | 16 | 3.4 |
| fresh | | | |
| A. palaestinum, 70% EtOH, | 55 | 5 | 11 |
| baked | | | |
| A. palaestinum, 20% EtOH, | >200 | <3 | >70 |
| fresh | | | |
| A. palaestinum, 20% EtOH, | >200 | 0.3-.5 | >400-700 |
| dried | | | |
| A. palaestinum, 20% EtOH, | >200 | 0.3-0.5 | >400-700 |
| baked | | | |
| A. palaestinum, H₂O, dried | >200 | 5-10 | >30 |
| A. palaestinum, H₂O, baked | >200 | 3 | >67 |

As can be seen, the Arum palaestinum formulations have very low toxicity, with toxicity levels well above effective levels required to achieve antiviral activity. Moreover, antiviral activity is observed at relatively low dosages. Overall, Arum palaestinum formulations present a favorable selectivity index, indicating it would be effective against SARS-CoV-2 infection at relatively low dosages, and safe to administer even at high dosages.

1. A method of inhibiting viral replication in a human subject exposed to or at risk of exposure to SARS-CoV-2 virus, or suffering from COVID-19, comprising the step of administering to the subject a prophylactically or therapeutically effective amount of a formulation comprising Arum palaestinum, which said formulation inhibits replication of said SARS-CoV-2 virus in said subject.

2. The method of claim 1, wherein said Arum palaestinum is an aqueous ethanol extract of Arum palaestinum leaves, stem, and/or bulb.

3. The method of claim 1, said formulation being administered to said human subject by a route selected from the group consisting of oral, rectal, nasal, ophthalmic, and parenteral administrations.

4. The method of claim 1, said formulation being administered at a level of from about 5 to 2000 mg per day.

5. The method of claim 1, wherein said human subject is suffering from one or more symptoms of COVID-19 prior to said administration step.

6. The method of claim 1, wherein said human subject is asymptomatic for COVID-19 prior to said administration step.

7. The method of claim 1, wherein said human subject has a baseline amount of detectable SARS-CoV-2 virus prior to said administration step, wherein after said administration step, said baseline amount of detectable virus is reduced.

8. The method of claim 7, wherein the baseline amount of detectable virus is reduced by at least 20% within 72 hours after said administration step.

9. A pharmaceutical formulation comprising Arum palaestinum, wherein said formulation inhibits replication of said SARS-CoV-2 virus in a subject, for the treatment of a human subject suffering from COVID-19, said formulation comprising an aqueous ethanol extract of Arum palaestinum leaves, stem, and/or bulb.

10. The pharmaceutical formulation of claim 9, said formulation being in a solid dosage form consisting of Arum palaestinum extract powder in a capsule.

11. A method of inhibiting SARS-CoV-2 viral replication, comprising the step of contacting said virus with a formulation comprising Arum palaestinum.
